(19) United States
(12) Patent Application Publication
(10) Pub. No.: US 2026/0338970 A1
(43) Pub. Date: Oct. 29, 2026

(54) RADIATION DETECTOR AND POSITRON EMISSION TOMOGRAPHY APPARATUS

(71) Applicant: HAMAMATSU PHOTONICS K.K., Hamamatsu-shi (JP)

(72) Inventors: Kento HAKAMATA, Hamamatsu-shi (JP); Hidemoto YAMAUCHI, Hamamatsu-shi (JP)

(73) Assignee: HAMAMATSU PHOTONICS K.K., Hamamatsu-shi (JP)

(21) Appl. No.: 19/640,978

(22) Filed: Apr. 7, 2026

(30) Foreign Application Priority Data
Apr. 28, 2025 (JP) 2025-074532

(51) Int. Cl. G01T 1/20; A61B 6/03; A61B 6/04; A61B 6/46

(52) U.S. Cl. CPC G01T 1/2018; A61B 6/037; A61B 6/0407; A61B 6/463

(57) ABSTRACT

A radiation detector, etc., that can reduce man-hours and time as compared with a case where a scintillator is partitioned into respective segments by modified regions formed by laser irradiation, and that can identify a segment in which scintillation light is generated in the same manner as in a case where modified regions are formed by laser irradiation. Embodiments include radiation detector having: a first photodetector; a second photodetector; and an elongated scintillator disposed between the first photodetector and the second photodetector, one end surface of the elongated scintillator being optically coupled to the first photodetector, and the other end surface of the elongated scintillator being optically coupled to the second photodetector.

### INCORPORATION BY REFERENCE

This application is based upon and claims the benefit of priority from Japanese patent application No. 2025-074532, filed on Apr. 28, 2025, the disclosure of which is incorporated herein in its entirety by reference.

### BACKGROUND

The present disclosure relates to a radiation detector and a positron emission tomography apparatus.

A radiation detector is known that comprises a scintillator having two scintillator portions arranged in parallel with each other, the scintillator being configured such that light (scintillation light) is allowed to flow between one end surface of each of the two scintillator portions (a U-shaped scintillator) (see, for example, Japanese Patent No. 6726969). The scintillator portions of Japanese Patent No. 6726969 are partitioned into a plurality of segments by modified regions formed by laser irradiation (light scattering portions in which light transmittance differs depending on an incident angle). By forming the modified regions, a ratio of scintillation light reaching both end surfaces of the scintillator varies for each segment in which scintillation light is generated, and therefore it is possible to identify the segment in which scintillation light is generated by performing predetermined calculation based on outputs of light receiving elements optically coupled to the both end surfaces.

### SUMMARY

However, since an extremely large number of scintillators are generally used in a positron emission tomography apparatus (PET (Positron Emission Tomography) apparatus) to which a radiation detector is applied, there is a problem in that forming modified regions by laser irradiation in each scintillator requires an enormous number of man-hours and a long time (for example, laser processing for one system requires six months to one year).

The present disclosure has been made in order to solve such problems, and an object thereof is to provide a radiation detector and a positron emission tomography apparatus that can reduce man-hours and time as compared with a case where a scintillator is partitioned into respective segments by modified regions formed by laser irradiation, and that can identify a segment in which scintillation light is generated in the same manner as in a case where modified regions are formed by laser irradiation.

A radiation detector according to a first aspect of the embodiment includes: a first photodetector; a second photodetector; and an elongated scintillator disposed between the first photodetector and the second photodetector, one end surface of the elongated scintillator being optically coupled to the first photodetector, and the other end surface of the elongated scintillator being optically coupled to the second photodetector, wherein at least one narrow light absorbing material that absorbs a part of scintillation light generated in the scintillator is provided on an outer peripheral surface of the scintillator.

Alternatively, a positron emission tomography apparatus according to a second aspect of the embodiment includes: a subject holding bed configured to hold a subject to which a radioactive agent has been administered; radiation detectors arranged in a ring shape for detecting radiation emitted from the subject; means for identifying a position of a radiation emission site based on outputs of the radiation detectors; means for generating an image indicating the position of the radiation emission site; and a display device for displaying the image, wherein the radiation detector comprises: a first photodetector; a second photodetector; and an elongated scintillator disposed between the first photodetector and the second photodetector, one end surface of the elongated scintillator being optically coupled to the first photodetector, and the other end surface of the elongated scintillator being optically coupled to the second photodetector, wherein at least one narrow light absorbing material that absorbs a part of scintillation light generated in the scintillator is provided on an outer peripheral surface of the scintillator.

As described above, according to the first aspect or the second aspect described above, it is possible to reduce man-hours and time as compared with a case where a scintillator is partitioned into respective segments by modified regions formed by laser irradiation, and to identify a segment in which scintillation light is generated in the same manner as in a case where modified regions are formed by laser irradiation.

Further, according to the above first aspect or the above second aspect, the scintillator is a U-shaped scintillator.

Further, according to the above first aspect or the above second aspect, the scintillator includes a plurality of segments partitioned by the light absorbing material, and wherein the light absorbing material is a light absorbing material in which a valley between the segments adjacent to each other appears in a DOI separation characteristic histogram.

Further, in the above first aspect or the above second aspect, the light absorbing material may be black tape, black coating, anodizing, a visible-light absorbing material, or a light absorbing film.

A radiation detector of the embodiment is a radiation detector that comprises [1]“a first photodetector; a second photodetector; and an elongated scintillator disposed between the first photodetector and the second photodetector, one end surface of the elongated scintillator being optically coupled to the first photodetector, and the other end surface of the elongated scintillator being optically coupled to the second photodetector, wherein at least one narrow light absorbing material that absorbs a part of scintillation light generated in the scintillator is provided on an outer peripheral surface of the scintillator.”

The radiation detector of the embodiment may be [2]“a radiation detector that is the radiation detector described in [1], wherein the scintillator is a U-shaped scintillator.”

The radiation detector of the embodiment may be [3]“a radiation detector that is the radiation detector described in [1] or [2], wherein the scintillator includes a plurality of segments partitioned by the light absorbing material, and wherein the light absorbing material is a light absorbing material in which a valley between the segments adjacent to each other appears in a DOI separation characteristic histogram.”

The radiation detector of the embodiment may be [4]“a radiation detector that is the radiation detector described in [3], wherein the light absorbing material is black tape, black coating, anodizing, a visible-light absorbing material, or a light absorbing film.”

A positron emission tomography apparatus of the embodiment may be [5]“a positron emission tomography apparatus that comprises: a subject holding bed configured to hold a subject to which a radioactive agent has been administered; radiation detectors arranged in a ring shape for detecting radiation emitted from the subject; means for identifying a position of a radiation emission site based on outputs of the radiation detectors; means for generating an image indicating the position of the radiation emission site; and a display device for displaying the image, wherein the radiation detector comprises: a first photodetector; a second photodetector; and an elongated scintillator disposed between the first photodetector and the second photodetector, one end surface of the elongated scintillator being optically coupled to the first photodetector, and the other end surface of the elongated scintillator being optically coupled to the second photodetector, wherein at least one narrow light absorbing material that absorbs a part of scintillation light generated in the scintillator is provided on an outer peripheral surface of the scintillator.”

According to the present disclosure, it is possible to provide a radiation detector and a positron emission tomography apparatus that can reduce man-hours and time as compared with a case where a scintillator is partitioned into respective segments by modified regions formed by laser irradiation, and that can identify a segment in which scintillation light is generated in the same manner as in a case where modified regions are formed by laser irradiation.

The above and other objects, features and advantages of the present disclosure will become more fully understood from the detailed description given hereinbelow and the accompanying drawings.

### BRIEF DESCRIPTION OF DRAWINGS

FIG. 1 is a schematic configuration diagram of the radiation detector 1 according to Embodiment 1;

FIG. 2 is a perspective view of a scintillator 10 and a light detection unit 20 (partially) extracted from FIG. 1;

FIG. 3 is a histogram (DOI separation characteristic histogram) summarizing calculation results of the DOI information;

FIG. 4 is a an example of a positron emission tomography apparatus 60 to which the radiation detector 1 of the present disclosure is applied;

FIG. 5 is an example of a positron emission tomography apparatus 60 to which the radiation detector 1 of the present disclosure is applied;

FIG. 6A is an example in which black tape BT is attached only to left and right side surfaces of a scintillator 10, which are a part of an outer peripheral surface;

FIG. 6B is an example in which black tape BT is attached only to a front surface of a scintillator 10, which is a part of an outer peripheral surface;

FIG. 6C is an example in which black tape BT is attached only to left and right side surfaces of a scintillator 10, which are a part of an outer peripheral surface;

FIG. 6D is an example of a scintillator 10 partitioned into three layers by attaching black tape BT to a part of an outer peripheral surface of the scintillator 10;

FIG. 7 is a schematic diagram of a scintillator 10A that is a modification; and

FIG. 8 is an example in which the light absorbing member 12 and the reflective member 13 are shared between adjacent scintillators 10.

### DETAILED DESCRIPTION OF EMBODIMENTS

### Embodiment 1

Hereinafter, a radiation detector 1 according to Embodiment 1 of the present disclosure will be described with reference to the accompanying drawings.

In the drawings, corresponding components are denoted by the same reference numerals, and redundant descriptions thereof are omitted.

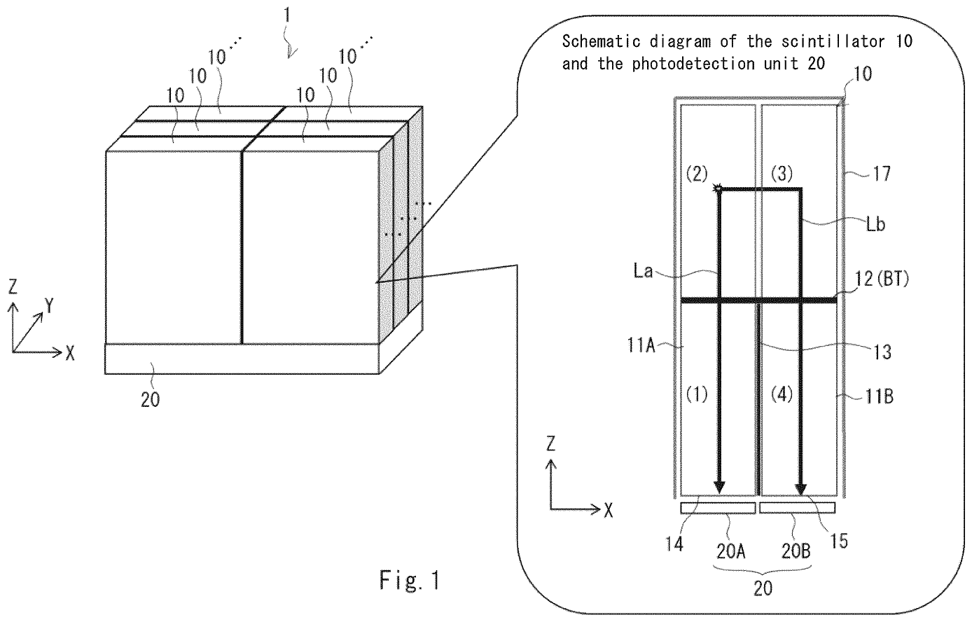

FIG. 1 is a schematic configuration diagram of the radiation detector 1 according to Embodiment 1.

The radiation detector 1 is a DOI (Depth of Interaction) detector that identifies a position (a position in a depth direction) at which scintillation light is generated due to interaction of high-energy gamma rays (annihilation radiation) incident on the scintillator 10 within the scintillator 10. As illustrated in FIG. 1, the radiation detector 1 includes a plurality of scintillators 10 and a light detection unit 20. The plurality of scintillators 10 are arranged in a matrix in an XY direction. Each scintillator 10 is covered with a reflective member 16 in order to prevent scintillation light from traveling between adjacent scintillators 10.

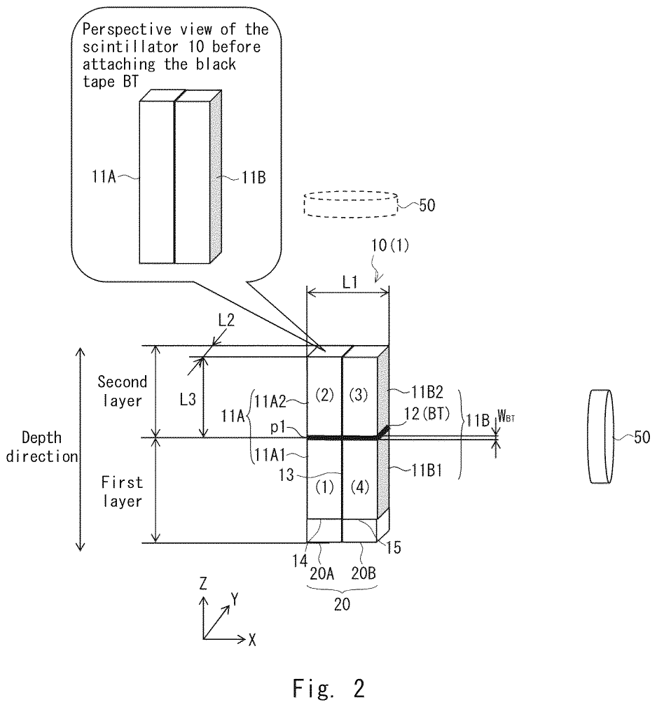

FIG. 2 is a perspective view of a scintillator 10 and a light detection unit 20 (partially) extracted from FIG. 1. For convenience of explanation, a reflective member 16 is omitted in FIG. 2. For convenience of explanation, as illustrated in FIG. 2 and the like, an X-axis, a Y-axis, and a Z-axis are defined. The X-axis extends in a left-right direction in FIG. 2. The Z-axis extends in an up-down direction in FIG. 2. The Y-axis extends in a direction orthogonal to an XZ plane.

As shown in FIG. 2, the scintillator 10 is a longitudinal scintillator (a U-shaped scintillator) disposed between a light receiving element 20A (an example of a first photodetector of the present disclosure) and a light receiving element 20B (an example of a second photodetector of the present disclosure), in which one end portion is optically coupled to the light receiving element 20A and the other end portion is optically coupled to the light receiving element 20B.

Specifically, the scintillator 10 includes a first scintillator portion 11A and a second scintillator portion 11B, which are arranged in parallel with each other. The material of the scintillator 10 is, for example, a crystal (crystal lump) such as Bi₄Ge₃O₁₂ (BGO), Ce-doped Lu₂SiO₅ (LSO), Lu₂(1−x)Y₂xSiO₅ (LYSO), Gd₂SiO₅ (GSO), Pr-doped LuAG (Lu₃Al₅O₁₂), Ce-doped LaBr₃ (LaBr₃), Ce-doped LaCl₃ (LaCl₃), or Ce-doped Lu0.7Y0.3AlO₃ (LuYAP), Lutetium Fine Silicate (LFS), and the like.

The first scintillator portion 11A and the second scintillator portion 11B will be described. Since the first scintillator portion 11A and the second scintillator portion 11B have the same configuration, hereinafter, the first scintillator portion 11A will be described as a representative.

The first scintillator portion 11A is a scintillator that is elongated in the Z direction. Specifically, the first scintillator portion 11A is a rectangular column shape scintillator including a pair of surfaces parallel to an XY plane, a pair of surfaces parallel to an XZ plane, and a pair of surfaces parallel to a YZ plane. Note that the first scintillator portion 11A may have a polygonal column shape other than the quadrangular prism shape. All surfaces of the first scintillator portion 11A are flatly polished. This polishing may be mechanical polishing or chemical polishing.

The first scintillator portion 11A and the second scintillator portion 11B are adjacently arranged in an X direction with their respective side surfaces (surfaces parallel to a YZ plane) facing each other. At this time, the first scintillator portion 11A and the second scintillator portion 11B are arranged such that, when projected onto the YZ plane, their respective outer shapes coincide on the YZ plane.

As shown in FIG. 2, at least one narrow light absorbing material 12 configured to absorb a part of scintillation light generated in the scintillator 10 is provided on an outer peripheral surface of the scintillator 10. Hereinafter, an example in which black tape BT is used as the light absorbing material 12 will be described. The black tape BT is, for example, an industrial tape CP743 manufactured by Shurtape Corporation. A width WBT of the black tape BT is about 1 mm. An attachment position p1 of the black tape BT is a central position of the scintillator 10 in a Z direction. The black tape BT is attached over an entire circumference of the outer peripheral surface at the attachment position p1.

By the black tape BT attached as described above, the first scintillator portion 11A is partitioned into a first-layer scintillator segment 11A1 (hereinafter also referred to as segment (1)) and a second-layer scintillator segment 11A2 (hereinafter also referred to as segment (2)). Similarly, the second scintillator portion 11B is partitioned into a first-layer scintillator segment 11B1 (hereinafter also referred to as segment (3)) and a second-layer scintillator segment 11B2 (hereinafter also referred to as segment (4)).

A reflective member 13 is disposed between segment (1) and segment (4) mainly to prevent scintillation light from traveling therebetween. The reflective member 13 is, for example, an ESR (Enhanced Specular Reflector) reflective material.

On the other hand, an optical discontinuity portion (here, an air layer) is provided between segment (2) and segment (3). The optical discontinuity portion may be, for example, an optical adhesive layer, instead of an air layer. It should be noted that no optical discontinuity portion is provided between segment (1) and segment (2). That is, the first scintillator portion 11A is a single crystal (crystal block), and segment (1) and segment (2) are optically continuous with each other. Similarly, no optical discontinuity portion is provided between segment (3) and segment (4). That is, the second scintillator portion 11B is a single crystal (crystal block), and segment (3) and segment (4) are optically continuous with each other.

In the scintillator 10 having the above configuration, that is, among scintillation light generated in any of segments (1) to (4), a part of scintillation light La (see FIG. 1), when passing between segments (1) and (2), has a portion thereof absorbed by a black tape BT that partitions the segments (1) and (2), and thereafter propagates to one end surface 14 of the first scintillator portion 11A parallel to an XY plane, and is incident on a light receiving element 20A optically coupled to the one end surface 14. Similarly, among scintillation light generated in any of segments (1) to (4), another part of scintillation light Lb (see FIG. 1), when passing between segments (3) and (4), has a portion thereof absorbed by a black tape BT that partitions the segments (3) and (4), and thereafter propagates to the other end surface 15 of the second scintillator portion 11B parallel to the XY plane, and is incident on a light receiving element 20B optically coupled to the other end surface 15. The light receiving elements 20A and 20B each output an electrical signal corresponding to light (photons) incident thereon.

Next, a method for identifying a segment in which scintillation light is generated will be described.

First, as illustrated in FIG. 2, a radiation source 50 that emits radiation in a 360-degree direction on a side of the scintillator 10 or facing the scintillator 10 (for example, a disk radiation source using radioactive isotope sodium-22) is disposed, and each time scintillation light is generated during a predetermined period (about 3 minutes), DOI information is calculated using the following Equation (1) and stored in a storage device (not illustrated).

$$\begin{matrix}[ Math . 1 ] & \\ DOI = \frac{B}{A + B} & Equation ( 1 )\end{matrix}$$

However, DOI represents DOI information, A represents an output value of the light receiving element 20A, and B represents an output value of the light receiving element 20B.

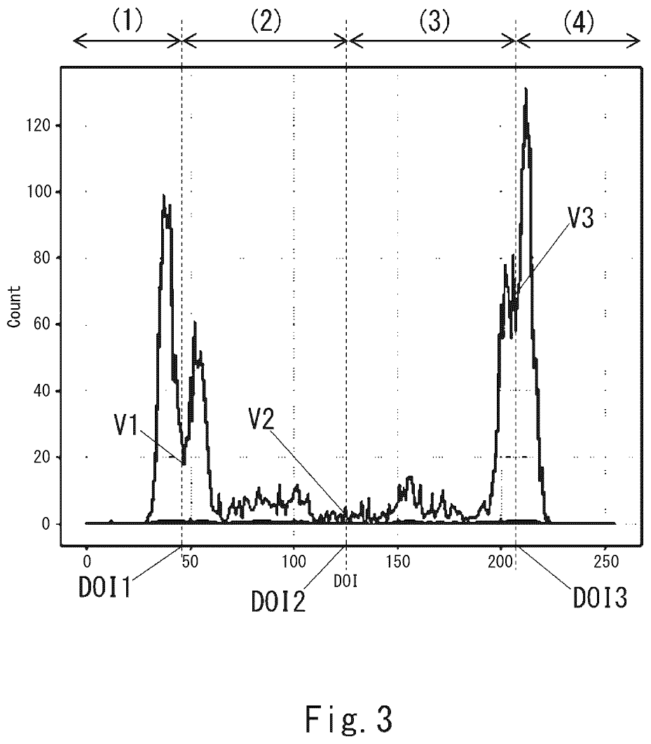

FIG. 3 is a histogram (DOI separation characteristic histogram) summarizing calculation results of the DOI information. In FIG. 3, a vertical axis represents the number of event occurrences, and a horizontal axis represents the DOI information.

Referring to FIG. 3, it can be seen that valleys V1, V2, and V3 exist between segment (1) and segment (2), between segment (2) and segment (3), and between segment (3) and segment (4), respectively. In FIG. 3, reference signs DOI1, DOI2, and DOI3 represent DOI information corresponding to valleys V1, V2, and V3, respectively.

The DOI information can be calculated using the above Equation (1) each time scintillation light is generated and outputs of the light receiving elements 20A and 20B are obtained. By comparing the calculated DOI information with DOI information DOI1 to DOI3 (threshold values), a segment in which scintillation light is generated can be identified.

For example, when the calculated DOI information is less than DOI1, a segment in which scintillation light is generated can be identified as segment (1). Similarly, when DOI1 is less than the calculated DOI information and the calculated DOI information is less than DOI2, the segment in which scintillation light is generated can be identified as segment (2). Similarly, when DOI2 is less than the calculated DOI information and the calculated DOI information is less than DOI3, the segment in which scintillation light is generated can be identified as segment (3). Similarly, when DOI3 is less than the calculated DOI information, the segment in which scintillation light is generated can be identified as segment (4). As a result of studies conducted by the inventors, it has been found that, when no black tape BT is attached to an outer peripheral surface of the scintillator 10, valleys between segments (in particular, valleys V1 and V3 shown in FIG. 3) do not clearly appear, and thus it becomes impossible to identify a segment in which scintillation light is generated.

As described above, by attaching the black tape BT to the outer peripheral surface of the scintillator 10, it becomes possible to identify the segment in which scintillation light is generated (that is, DOI separation becomes possible). This is because, by attaching the black tape BT, which is a light absorbing material, to the outer peripheral surface of the scintillator 10 so as to partition the respective segments, the ratio of amounts of scintillation light incident on the light receiving elements 20A and 20B changes, in the same manner as in the above-described prior art in which the respective segments are partitioned by modified regions formed by laser irradiation.

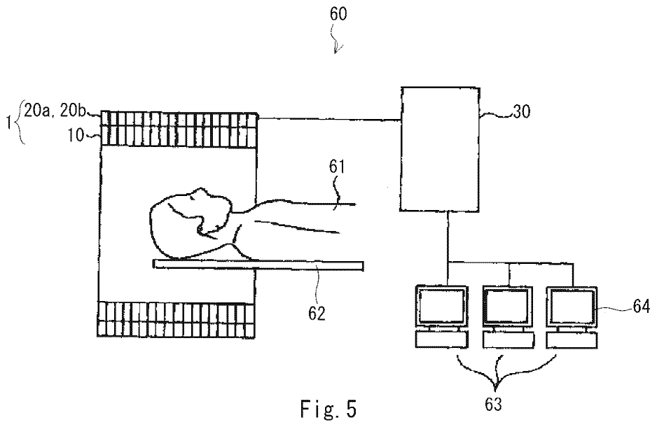

The light detection unit 20 includes a first light receiving element 20A optically coupled to the end surface 14 of the first scintillator portion 11A, and a second light receiving element 20B optically coupled to the end surface 15 of the second scintillator portion 11B. The light receiving elements 20A and 20B are, for example, semiconductor photodetectors using MPPCs (Multi-Pixel Photon Counters). An MPPC is a photon-counting device composed of a plurality of Geiger-mode APD pixels. The light receiving elements 20A and 20B output electrical signals corresponding to light (photons) incident thereon. These electrical signals are input to a signal processing circuit 30 (see FIG. 5).

Next, a positron emission tomography apparatus 60 (PET (Positron Emission Tomography) apparatus) to which the radiation detector 1 of the present disclosure is applied will be briefly described.

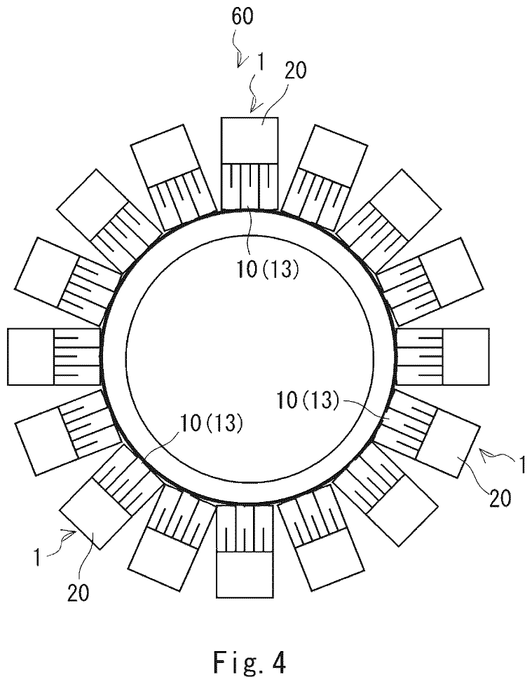

FIGS. 4 and 5 illustrate an example of a positron emission tomography apparatus 60 to which the radiation detector 1 of the present disclosure is applied.

As shown in FIGS. 4 and 5, the positron emission tomography apparatus 60 includes a subject holding bed 62 that holds a subject 61 to which a radiopharmaceutical has been administered, a plurality of radiation detectors 1 arranged in a ring shape to detect radiation (annihilation gamma rays) generated from the subject 61, means for identifying a position of a radiation emission site based on outputs of the radiation detectors 1 (the light detection unit 20) (for example, a signal processing circuit 30), means for generating an image indicating the position of the emission site (for example, an information processing device 63 such as a personal computer) and a display device 64 configured to display the image indicating the position of the emission site.

The signal processing circuit 30 executes processing for identifying a position (segment) in which scintillation light is generated, that is, a position of a radiation emission site, by comparing the DOI information calculated as described above with DOI information DOI1 to DOI3 (threshold values) stored in a storage device (not illustrated). An output of the signal processing circuit 30 is input to the information processing apparatus 63.

The information processing apparatus 63 generates an image indicating a position of a radiation emission site based on an output of the signal processing circuit 30, that is, a segment in which scintillation light is generated (a position of the radiation emission site), and displays the generated image on the display device 64.

As described above, according to the present embodiment, by partitioning the scintillator 10 into respective segments using the black tape BT, which is a light absorption material, it is possible to reduce the number of process steps and processing time as compared with a case where the scintillator is partitioned into respective segments by a modified region formed by laser irradiation, and to identify a segment in which scintillation light is generated in the same manner as in a case where the modified region formed by laser irradiation is provided.

Next, a modification example will be described.

In the above embodiment, an example has been described in which the attachment position p1 of the black tape BT (see FIG. 2) is at the center position of the scintillator 10 in the Z direction, and the width WBT (see FIG. 2) is about 1 mm; however, the present disclosure is not limited thereto. That is, the attachment position p1 and the width WBT of the black tape BT may be appropriately adjusted so that valleys (in particular, valleys V1 and V3 shown in FIG. 3) clearly appear. The same applies to the reflectance of the black tape BT.

For example, the attachment position p1 of the black tape BT may be a position shifted in the Z direction from the center position of the scintillator 10 in the Z direction. On the other hand, if the width WBT of the black tape BT is made excessively large, the amount of scintillation light incident on the light receiving elements 20A and 20B decreases, making it impossible to identify the segment in which scintillation light is generated. Therefore, it is desirable that the width WBT of the black tape BT be about 1 mm or 1 mm or less, in consideration of the amount of scintillation light incident on the light receiving elements 20A and 20B and the DOI separation characteristics.

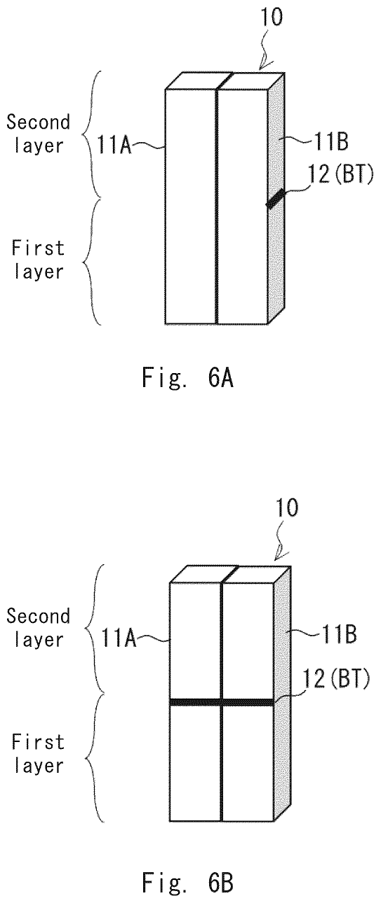

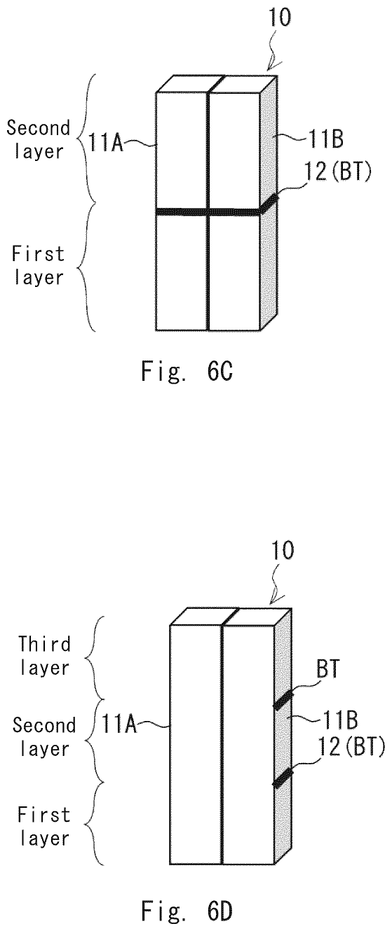

In the above embodiment, an example has been described in which the black tape BT is attached over the entire circumference of the outer peripheral surface; however, the present disclosure is not limited thereto. That is, as long as valleys (in particular, valleys V1 and V3 shown in FIG. 3) appear clearly, the black tape BT may be attached to only a part of the outer peripheral surface, rather than the entire circumference, as shown in FIGS. 6(a) to 6(c). For example, as shown in FIG. 6(a), the black tape BT may be attached only to the left and right side surfaces of the scintillator 10, which are parts of the outer peripheral surface. Alternatively, as shown in FIG. 6(b), the black tape BT may be attached only to the front surface of the scintillator 10, which is a part of the outer peripheral surface. Further, as shown in FIG. 6(c), the black tape BT may be attached only to the left and right side surfaces and the front surface of the scintillator 10, which are parts of the outer peripheral surface. Although not illustrated, the black tape BT may be attached only to the rear surface of the scintillator 10, or only to the front surface and the rear surface thereof, or to other part(s) of the outer peripheral surface.

In the above embodiment, an example has been described in which the scintillator 10 is divided into two layers by attaching the black tape BT; however, the present disclosure is not limited thereto. For example, by attaching the black tape BT, the scintillator 10 may be divided into three layers (see FIG. 6(d)) or into a multilayer structure having more layers. FIG. 6(d) illustrates an example of a scintillator 10 that is divided into three layers by attaching the black tape BT to a part of the outer peripheral surface of the scintillator 10.

In the above embodiment, an example has been described in which the black tape BT, which is a light absorption material, is applied to the U-shaped scintillator 10 shown in FIG. 2; however, the present disclosure is not limited thereto. That is, the black tape BT may be applied not only to the U-shaped scintillator 10 but also to any scintillator, as long as the scintillator is an elongated scintillator disposed between a first photodetector and a second photodetector, with one end portion optically coupled to the first photodetector and the other end portion optically coupled to the second photodetector.

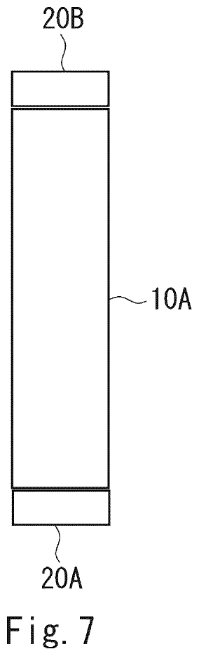

For example, as illustrated in FIG. 7, the present disclosure may also be applied to a both-end readout DOI configuration that uses a linear scintillator 10A and light receiving elements 20A and 20B disposed at respective ends of the scintillator. FIG. 7 is a schematic diagram of a scintillator 10A according to a modification.

In the above-described embodiment, an example has been described in which black tape BT is used as the light absorbing member 12; however, the present disclosure is not limited thereto. For example, other light absorbing materials such as black paint, anodized coatings, visible-light absorbing materials, or light absorbing films may be used as the light absorbing member 12. The position, width, reflectance, and the like of such other light absorbing materials (e.g., black paint) may be the same as those of the black tape BT described above.

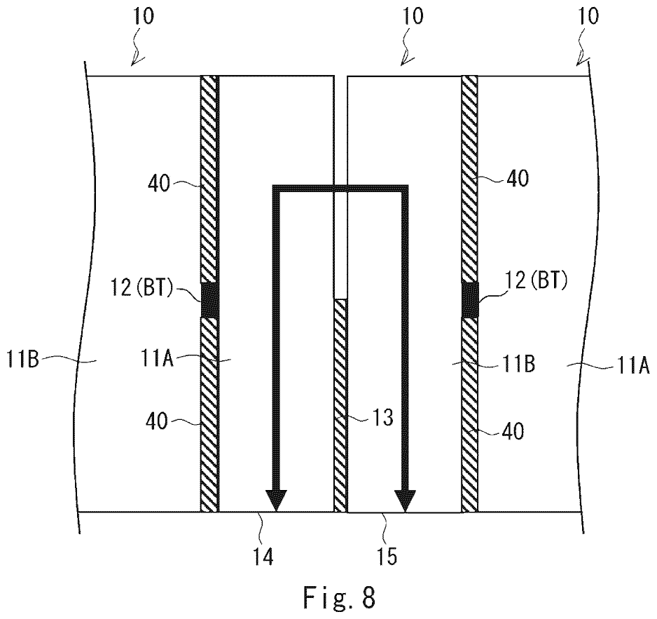

When a light absorbing member 12 having a certain thickness, such as the black tape BT, is used, the light absorbing member 12 and the reflective member 13 may be provided between adjacent scintillators 10, as illustrated in FIG. 8. FIG. 8 illustrates an example in which the light absorbing member 12 and the reflective member 13 are shared between adjacent scintillators 10. In this case, it is preferable that the light absorbing member 12 and the reflective member 13 are in close contact with each of the adjacent scintillators 10.

All numerical values presented in the above embodiments are illustrative only, and it goes without saying that other appropriate numerical values different therefrom may be employed.

The above embodiments are merely examples in all respects. The present disclosure should not be construed as being limited by the description of the above embodiments. The present disclosure may be embodied in various other forms without departing from the spirit or essential characteristics thereof.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A radiation detector comprising:
a first photodetector;
a second photodetector; and
an elongated scintillator disposed between the first photodetector and the second photodetector, one end surface of the elongated scintillator being optically coupled to the first photodetector, and another end surface of the elongated scintillator being optically coupled to the second photodetector,
wherein at least one narrow light absorbing material that absorbs a part of scintillation light generated in the scintillator is provided on an outer peripheral surface of the scintillator.

2. The radiation detector according to claim 1, wherein the scintillator is a U-shaped scintillator.

3. The radiation detector according to claim 1,
wherein the scintillator includes a plurality of segments partitioned by the light absorbing material, and
wherein the light absorbing material is a light absorbing material in which a valley between the segments adjacent to each other appears in a depth of interaction (DOI) separation characteristic histogram.

4. The radiation detector according to claim 3,
wherein the light absorbing material is black tape, black coating, anodizing, a visible-light absorbing material, or a light absorbing film.

5. A positron emission tomography apparatus comprising:
a subject holding bed configured to hold a subject to which a radioactive agent has been administered;
radiation detectors arranged in a ring shape for detecting radiation emitted from the subject;
means for identifying a position of a radiation emission site based on outputs of the radiation detectors;
means for generating an image indicating the position of the radiation emission site; and
a display device for displaying the image,
and the radiation detector comprises:
a first photodetector;
a second photodetector; and
an elongated scintillator disposed between the first photodetector and the second photodetector, one end surface of the elongated scintillator being optically coupled to the first photodetector, and another end surface of the elongated scintillator being optically coupled to the second photodetector,
wherein at least one narrow light absorbing material that absorbs a part of scintillation light generated in the scintillator is provided on an outer peripheral surface of the scintillator.
